(19) United States
(12) Patent Application Publication
(10) Pub. No.: US 2008/0214924 A1
(43) Pub. Date: Sep. 4, 2008

(54) Magnetic Resonance Spectroscopy

(75) Inventors: Marinus Johan Kruiskamp, Zaltbommel (NL); Arianne Van Muiswinkel, Best (NL)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 11/995,933

(22) PCT Filed: Jul. 19, 2006
(86) PCT No.: PCT/IB2006/052468

§ 371 Date: Jan. 17, 2008

(30) Foreign Application Priority Data
Jul. 20, 2005 (EP) 05106623.1

(51) Int. Cl. A61B 5/055

(52) U.S. Cl. 600/410

(57) ABSTRACT

A magnetic resonance (MR) imaging (MRI) system (101) comprising a magnet system (102) for acquiring MR spectroscopic data from an object of interest (103) comprised within a polarizing magnetic field is described, wherein the MRI system (101) comprises multiple pole faces (104) for generating the polarizing magnetic field. The multiple pole faces (104) provide a gap into which the object of interest (103), for example, a human patient or a dummy or an imaging phantom may be introduced. The gap is typically large enough to allow interventional procedures to be performed in between the pole faces (104) of the magnet (102).

The invention relates to a magnetic resonance (MR) imaging system comprising a magnet system for acquiring MR spectroscopic data from an object of interest comprised within a polarizing magnetic field.

The invention further relates to a method of acquiring MR spectroscopic data, the method comprising the steps of generating a polarizing magnetic field and acquiring MR spectroscopic data from an object of interest comprised within the polarizing magnetic field.

The invention further relates to a computer program product to be loaded by a computer arrangement comprising a processing unit and a memory, the computer program product comprising instructions for acquiring MR spectroscopic data from an MRI system and, after being loaded, enabling the MRI system to carry out said method.

Magnet systems can be broadly categorized into “bore-type” and “gap-type” systems, also referred to as “cylindrical” and “open” systems, respectively. In a bore-type system, a solenoid magnet generates a polarizing magnetic field, B₀, along its own longitudinal axis and the common longitudinal axis of the cylindrical bore of the system. In a gap-type system, the B₀ field is generated in an open region between a pair of magnetic pole faces. In either case, the direction of the B₀ field is commonly denoted as the z-axis. The polarizing magnetic field B₀ polarizes the nuclear magnetic spin system of an object placed within the field. To generate an MR signal, the polarized spin system is first excited by applying an MR excitation signal or radio frequency (RF) magnetic field B₁, perpendicular to the z-axis. The RF excitation pulse tips the magnetization out of alignment with the z-axis and causes its macroscopic magnetic moment vector to precess around the z-axis. The precessing magnetic moment, in turn, generates an RF MR signal in a pick-up or receiver coil placed perpendicular to the z-axis.

To generate an MR image, gradient pulses are typically applied along the x, y and z-axis directions to localize the spins along the three spatial dimensions, and MR signals are acquired in the presence of one or more readout gradient pulses. An image depicting the spatial distribution of a particular nucleus in a region of interest of the object is then generated, using known post-processing techniques. Typically, the hydrogen nucleus (¹H) is imaged, though other MR-detectable nuclei may also be used to generate images.

In the case of MR spectroscopy, MR signals are acquired in the absence of readout gradient pulses, though other gradient pulses may be used to prepare the spins prior to signal acquisition. The acquired MR signals are processed and normally displayed in the form of spectra that contain information based on the differences in the resonance frequencies of different MR-detectable nuclei. In practice, different signals from MR-detectable nuclei of a single species or element resonating at slightly different resonance frequencies are acquired to generate the spectra. The slight difference in resonance frequencies arises from the nuclei existing in different chemical environments. This frequency difference is measured in units of parts per million (ppm), as it is of the order of a few Hertz (Hz) as compared to the MR signals which precess at the rate of a few million Hz.

To be able to delineate the various MR signals arising a few ppm apart, MR spectroscopic systems benefit from higher magnetic field strengths. Such systems also impose more stringent requirements on the homogeneity of the B₀ field. Bore-type magnets typically have higher field strengths and a better magnetic field homogeneity as compared to gap-type systems. Hence, there is a prejudice in the prior art against the use of gap-type systems for MR spectroscopy. In addition, there is also a prejudice against the use of MR imaging systems with a polarizing field strength of less than 1.5 T for MR spectroscopy.

Prejudice against the use of gap-type magnet systems and low-field and mid-field MRI systems for MR spectroscopy may be found in an article by Melissa Minkin, entitled “Open vs. Closed MR: Options for Brain Tumor Patients”, in the magazine “Search”, Issue No. 47, Spring 2001, which states: “ . . . ” open MRI still lacks the ability to perform advanced techniques like a Functional MRI exam”. The next paragraph continues with: “Magnetic resonance spectroscopy (MRS) is another technique reserved for high field systems”.

Additional evidence of prejudice is found in the publication, “Susil R C, Menard C, et al., Transrectal Prostate Biopsy and Fiducial Marker Placement in a Standard 1.5 T MRI Scanner, Journal of Radiology 2004 (in review)”, which states: “Prior work has been performed on low field strength (e.g. 0.2 or 0.5 T) open scanner architectures (6-8). While these systems provide easier access to the patient, they do not produce the highest quality MR images, have limited potential for functional and spectroscopic imaging, and are less widely available.”

The Scottish Health Guidance Note on Magnetic Resonance Imaging, by the NHSScotland Property & Environment Forum, June 2001, states: “High field MRI systems may also be capable of performing magnetic resonance spectroscopy, which provides in-vivo biochemical information on cell metabolism”. At a later point in the document, “high field” is defined as “1.5 T or above”.

Further evidence as to the prejudice regarding the requirement of high field strengths for MR spectroscopy, clearly defined as 1.5 T or higher, is disclosed in EP085240A1.

EP085240A1 describes an embodiment of a main field magnet having a large clear material section and a low main magnetic field strength, wherein the main field magnet is combined with a small magnet which is capable of generating a high magnetic field. The small magnet has means for making it mobile so that it can be transported between an inoperative position outside the large magnet and an operative position inside the large magnet. The main field magnet is used for interventional MR imaging, while the small magnet is used for MR spectroscopy.

A problem with the prior art is that the small magnet, which is capable of MR spectroscopy, is not large enough to facilitate interventional procedures to be performed inside the magnet.

It is therefore an object of the invention to provide an MRI system which is capable of MR spectroscopy and allows interventional procedures to be performed inside the MRI system.

This object is achieved by an MRI system as described in the opening paragraph, wherein the MRI system comprises multiple pole faces for generating the polarizing magnetic field. The multiple pole faces provide a gap into which the object of interest, for example, a human patient or a dummy or an imaging phantom may be introduced. The gap is typically large enough to permit interventional procedures to be performed in between the pole faces of the magnet.

These and other aspects of the invention will be elaborated on the basis of the following preferred embodiments, which are defined in the dependent claims.

Since the magnetic field homogeneity requirements for MR spectroscopy are more stringent as compared to those for MRI, one preferred embodiment of the MRI system comprises an indicator for indicating a region for acquiring MR spectroscopic data. This region will hereinafter be referred to as the spectroscopic volume. The indicator may be, for example, an optical means, such as a set of laser beams, or a mechanical means, such as physical markers on the patient table, etc.

Another preferred embodiment comprises a positioner placed within the magnet system, to position the object of interest, for example, a selected part of a patient's body, an imaging phantom, an animal, or a dummy, in the spectroscopic volume. The positioner is preferably a constraining device that immobilizes the object of interest. The positioner may be alternatively a device that allows movement of or around the object of interest, for example, the neck, a knee, an elbow, or other parts of the body, while still retaining the object of interest within the spectroscopic volume. The positioner may be alternatively a resistance device that enables load-bearing or stress studies of the object of interest.

It is a further object of the invention to provide a method of acquiring MR spectroscopic data from an MRI system that allows interventional procedures to be performed in the MRI system.

This object is achieved by a method as described in the opening paragraphs, wherein the polarizing magnetic field is generated between multiple pole faces of the MRI system.

Further embodiments of the method are defined in the dependent claims 5 and 6.

It is a further object of the invention to provide a computer program product to be loaded by a computer arrangement, the computer program product comprising instructions for acquiring MR spectroscopic data from an MRI system that allows interventional procedures to be performed in the MRI system.

This object is achieved by a computer program product as described in the opening paragraphs, wherein the polarizing magnetic field is generated between multiple pole faces of the MRI system.

These and other aspects of the invention will be described in detail hereinafter, by way of example, on the basis of the following embodiments, with reference to the accompanying drawings, wherein

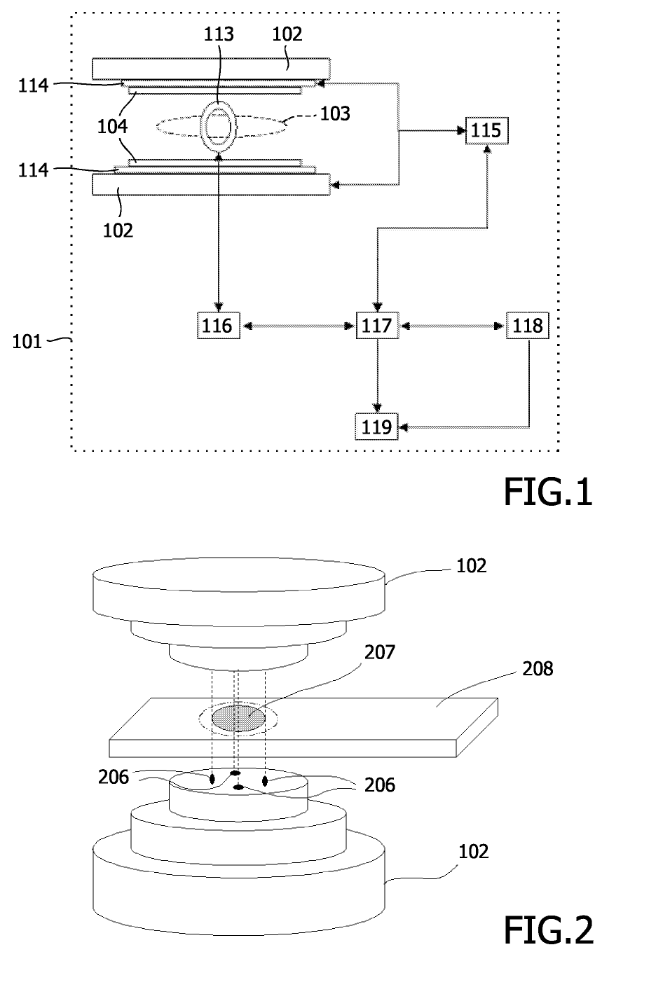

FIG. 1 is a block diagram of the setup according to the invention, in which a gap-type MRI system is arranged to acquire MR spectroscopic data from an object of interest comprised in the MRI system,

FIG. 2 schematically shows a preferred embodiment of the invention, in which different indicators indicate the boundaries of the spectroscopic volume comprised in the MRI system,

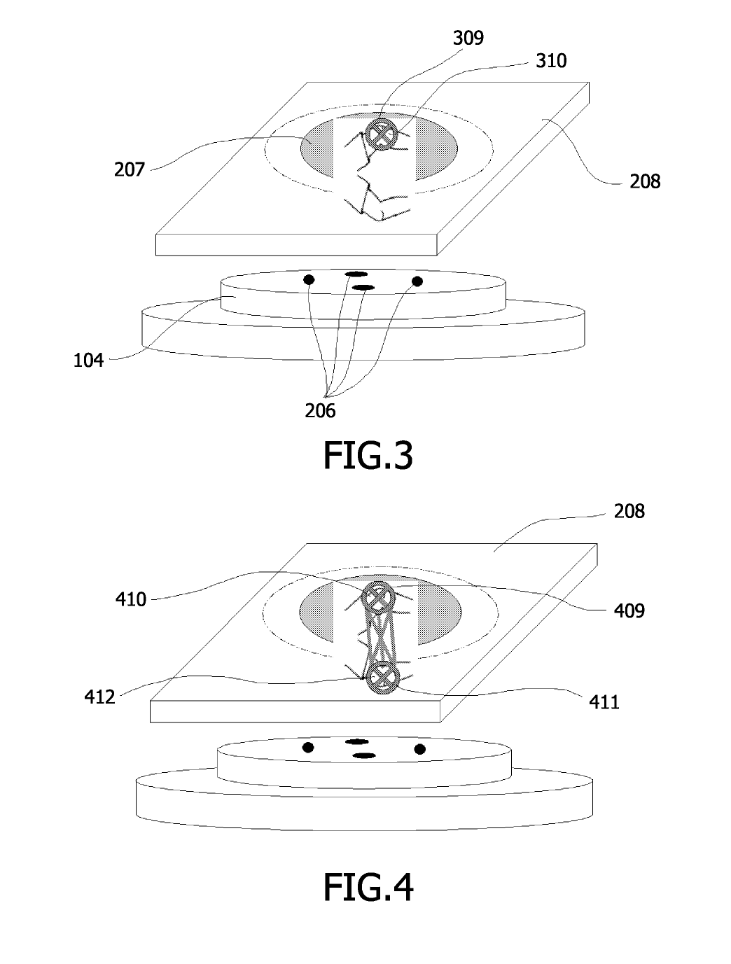

FIG. 3 schematically shows a preferred embodiment of the invention, in which a positioner is used to position at least a part of the object of interest in the spectroscopic volume comprised in the MRI system,

FIG. 4 schematically shows a preferred embodiment of the invention, in which multiple positioners are used to position multiple objects of interest in the spectroscopic volume comprised in the MRI system,

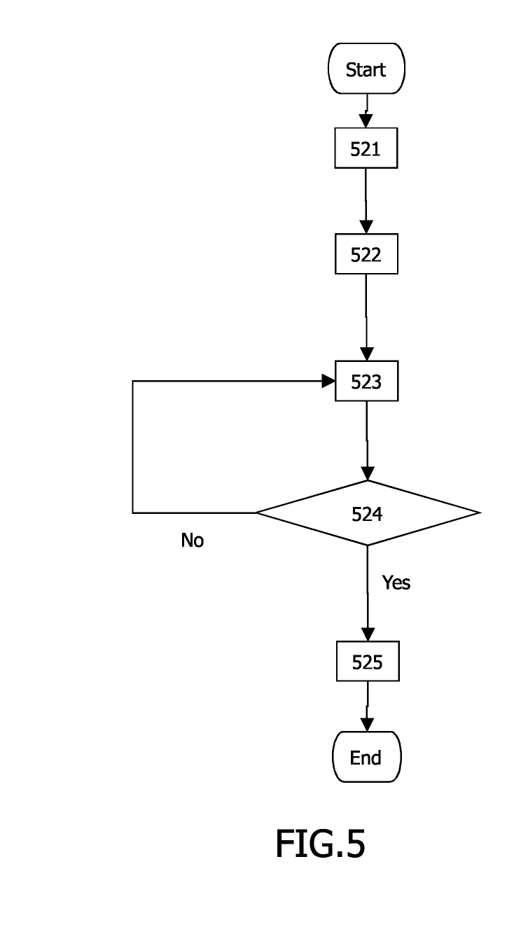

FIG. 5 schematically shows a preferred embodiment of the method of acquiring MR spectroscopic data from an object of interest comprised in a polarizing magnetic field of a gap-type MRI system,

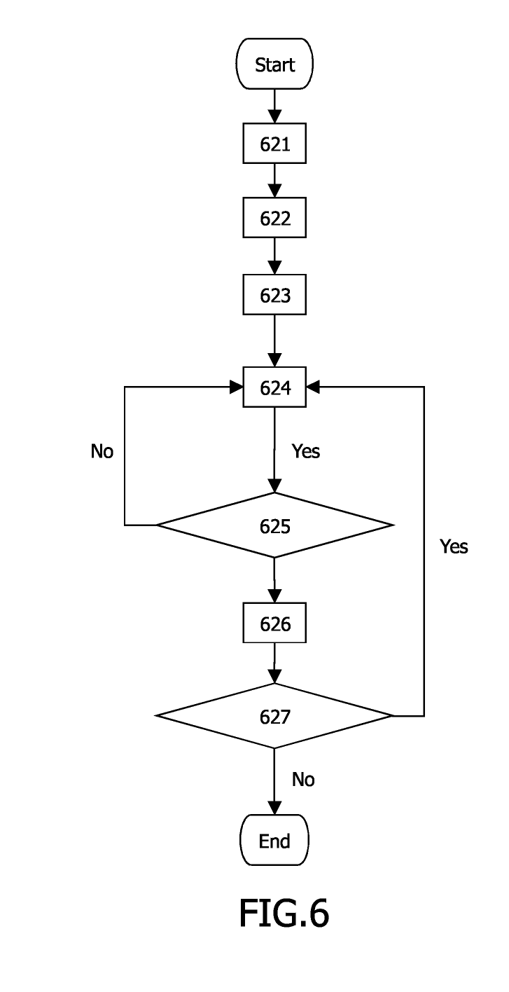

FIG. 6 schematically shows a preferred embodiment of the method of acquiring MR spectroscopic data from multiple regions of interest of an object of interest comprised in a polarizing magnetic field of a gap-type MRI system, and

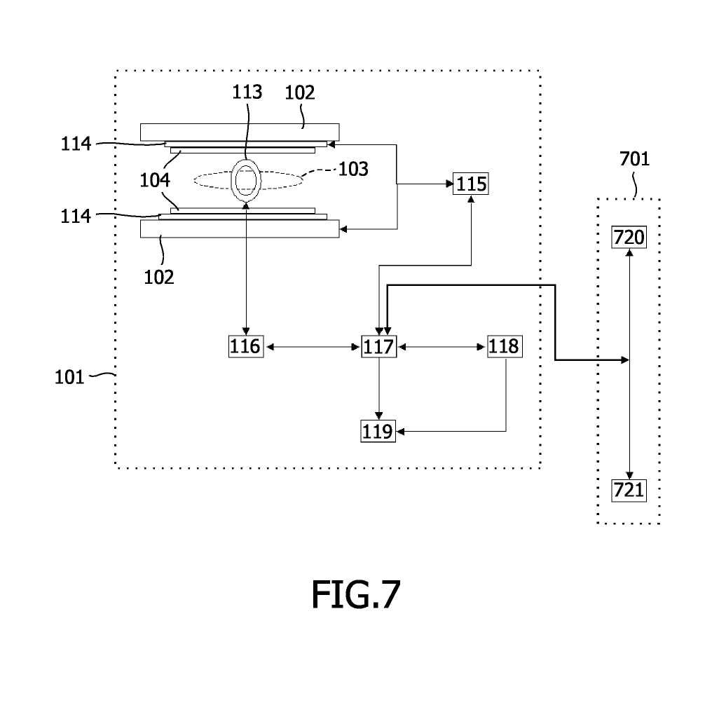

FIG. 7 schematically shows a preferred embodiment of a computer program product according to the invention.

It should be noted that corresponding reference numerals used in the various Figures represent corresponding structures in these Figures.

FIG. 1 is a block diagram of the setup according to the invention. The Figure shows a gap-type MRI system 101, also referred to as an open MRI system, comprising a magnet 102. The magnet 102 preferably has two pole faces 104 with an air gap in between them, into which an object of interest 103 may be introduced. The MRI system further comprises multiple gradient coils 114 connected to a gradient driver unit 115. The MRI system 101 also comprises transmitting and receiving RF coils 113 connected to an RF coil driver unit 116. A control unit 117 controls the operation of a reconstruction unit 118, a display unit 119, the gradient driver unit 115 and the RF coil driver unit 116.

The pole faces 104 generate a static magnetic field strength of, for example 1.0 T, in the air gap. The object of interest 103, for example, a human or an animal or an imaging phantom, is placed in the air gap. To enable MR imaging, temporally variable magnetic field gradients superimposed on the static magnetic field are generated by the multiple gradient systems 114 in response to currents supplied by the gradient driver unit 115. The control unit 117 controls the characteristics of the currents, notably their strengths, durations and directions, flowing through the gradient coils. The RF coils 113 generate RF excitation pulses in the object of interest 103 and receive MR signals generated by the object of interest 103 in response to the RF excitation pulses. For MR spectroscopy, free induction decay (FID) signals are typically received by the RF coil 113, while for MR imaging, gradient recalled echoes or spin echoes are typically received. The RF coil driver unit 116 supplies current to the RF coil 113 to transmit the RF excitation pulse, and amplifies the MR signals received by the RF coil 113. The characteristics of the transmitted RF excitation pulses, notably their strength and duration, are controlled by the control unit 117. It is to be noted that, although the transmitting and receiving coil are shown as one unit in this embodiment, it is also possible to have separate coils for transmission and reception, respectively. It is further possible to have multiple RF coils 113 for transmission or reception, or both. The RF coils 113 may be integrated into the magnet 102, or may be separate surface coils. The received signals are reconstructed by the reconstruction unit 118 and displayed on the display unit 119. FID signals are typically Fourier-transformed in one dimension by the reconstruction unit 118 and displayed as spectra, while echoes are Fourier-transformed in two dimensions and displayed as two-dimensional images.

An advantage of the gap-type system is that it enhances patient comfort due to the fact that somebody can be close to the object of interest, such as a patient, to give the patient a secure feeling during the data acquisition. This is especially advantageous while studying pediatric patients.

FIG. 2 shows an advantageous embodiment of the invention, in which indicators are provided to indicate the boundaries of the spectroscopic volume. The indicator may be an optical system 206 comprising, for example, multiple laser beams. Alternatively, they may be mechanical markers, or markings 207 on a transport system 208, which may be, for example, a patient table. The indicators 206, 207 may also be positioned on other parts of the MRI system, such as a typical C-arm (not shown) that connects the two magnets 102. Alternatively, the indicators may be part of a separate unit that may be removably attached to the MRI system. By appropriately distributing multiple indicators in three-dimensional space, for example, with one indicator arranged to indicate the boundary in the x-y plane and a second indicator arranged to indicate the boundary in the x-z plane, it is possible to demarcate the boundaries of the spectroscopic volume in all of the three spatial dimensions.

It is known that MR spectroscopy requires more homogeneous fields when compared to MR imaging. For example, MR imaging typically requires magnetic fields with a homogeneity of around 10 ppm, peak-to-peak, in a volume of typically around 50 cm³. MR spectroscopy typically requires a homogeneity of less than 1 ppm, peak-to-peak, although it may be in a smaller volume. This volume of optimal homogeneity for either spectroscopy or imaging is called the “sweet spot” for that particular application. It is known that the magnetic field homogeneity deteriorates when one moves away from the sweet spot. The indicators 206, 207 thus indicate the limits of the spectroscopic volume, or the “sweet spot” for spectroscopy. Such an indicator can be useful to an operator, for example, to confirm that, before starting data acquisition, the region of interest is contained within the spectroscopic volume.

The indicators 206, 207 are also moveable and provide a feedback of their current position to the MRI system 101, such that the system software can warn the operator if the region of interest of the object of interest 103 extends beyond the spectroscopic volume. When the operator positions the region of interest within the magnet 102, using available landmarking or referencing facilities, the optical, mechanical or other indicators 206, 207 can be moved to the limits of the region of interest, from which MR spectroscopic data is to be acquired. If the limits of the desired region of interest as specified by the indicators 206, 207 fall outside the spectroscopic volume, the system software can warn the operator to adjust the region of interest. Alternatively, the software can control the patient table 208 in such a way that the region extending beyond the spectroscopic volume is automatically brought within the spectroscopic volume before MR spectroscopic data are acquired from the region.

FIG. 3 shows a further advantageous embodiment of the invention, in which a positioner 309 is used to position at least a part of the object of interest, in this case, a knee 310, within the spectroscopic volume. Such a positioner 309 may be a set of straps immobilizing the region of interest and constraining it within the spectroscopic volume. Alternatively, the positioner 309 may be a device allowing mobility of the region of interest while still constraining it within the spectroscopic volume. For example, it allows motion of extremities, e.g. bending of arms, legs, shoulders, hips, etc. to image them in different positions. Furthermore, it also allows the subject to perform various exercises while positioned inside the magnet. MR spectroscopic data are collected prior to, during and after the exercise to study the effect of said exercise on various metabolites.

FIG. 4 shows a further advantageous embodiment, in which multiple positioners 409, 411 are used to constrain multiple regions of interest 410, 412. The MRI system can be programmed to automatically collect MR spectroscopic data from the various regions of interest 410, 412. For example, if the two positioners 409, 411 are used to position the two knees 410, 412 of a patient, the MRI system can be programmed to collect MR spectroscopic data first from one knee 410, then shift the patient table until the second positioner 411 (and hence, the second knee 412) is within the spectroscopic volume, and then collect MR spectroscopic data from the second knee 412. It is an advantage of the gap-type magnet system that the patient table may be moved laterally, in addition to or in combination with the axial movement that is possible on a normal MRI system. Hence, it is advantageous to conduct MR spectroscopic studies combined with interventional procedures on gap-type magnet systems, especially while studying laterally separated regions of interest, such as the two knees or the two shoulders of a human patient.

FIG. 5 shows an advantageous embodiment of the method of the invention, wherein the method comprises a step 521 of generating a polarizing magnetic field between multiple pole faces of an MRI system, a step 522 of positioning the object of interest within the polarizing magnetic field, a step 523 of adjusting the position of a region of interest to be comprised in the spectroscopic volume, a step 524 of checking whether the region of interest is comprised in the spectroscopic volume, and a step 525 of acquiring MR spectroscopic data from the region of interest. Steps 523 and 524 are repeated, if required, until the region of interest is comprised in the spectroscopic volume.

FIG. 6 shows an advantageous embodiment of the method of the invention for collecting spectroscopic data from multiple regions of interest, the method comprising a step 621 of generating a polarizing magnetic field between multiple pole faces of an MRI system, a step 622 of placing multiple regions of interest of the object of interest in multiple positioners, a step 623 of positioning a positioner comprising a region of interest within the polarizing magnetic field, a step 624 of adjusting the position of the positioner comprising the region of interest so that the region of interest is comprised in the spectroscopic volume, a step 625 of checking whether the region of interest is comprised in the spectroscopic volume, and a step 626 of acquiring MR spectroscopic data from the region of interest. The method is continued with a step 627 of checking whether there are additional regions of interest to be scanned. If it is determined that additional regions of interest need to be scanned, steps 624 and 625 are repeated until a subsequent positioner comprising a subsequent region of interest is comprised in the spectroscopic volume. Step 626 of acquiring spectroscopic data from the next region of interest is repeated. When the result of step 627 indicates that there are no more regions of interest to be scanned, the method is terminated.

FIG. 7 shows an embodiment of the computer program product to be loaded by a computer arrangement 701 comprising a computer processing unit 720 and a memory unit 721. The computer processing unit 720 generates and inputs instructions to a control unit 117, to enable an MRI system 101 to generate a polarizing magnetic field between multiple pole faces 104 of the MRI system 101, generate temporally variable gradient magnetic fields superimposed on the polarizing magnetic field, generate RF excitation pulses in an object of interest 103 comprised in the polarizing magnetic field, acquire MR spectroscopic or MR imaging data from an object of interest 103 comprised in the polarizing magnetic field, reconstruct the acquired MR spectroscopic or MR imaging data on the reconstruction unit 118 and display reconstructed data on the display unit 119.

The order of the described embodiments of the method of the invention is not mandatory. A person skilled in the art may change the order of steps or perform steps concurrently, using threading models, multi-processor systems or multiple processes without departing from the concept as intended by the invention.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the word “comprising” does not exclude the presence of elements or steps other than those listed in a claim. Use of the indefinite article “a” or “an” preceding an element does not exclude the presence of a plurality of such elements. The invention can be implemented by means of hardware comprising several distinct elements, and by means of a suitably programmed computer. In the system claims enumerating several means, several of these means can be embodied by one and the same item of computer-readable software or hardware. The mere fact that certain measures are recited in mutually different dependent claims does not indicate that a combination of these measures cannot be used to advantage.

It is to be further noted that the term “MRI system” as used in the application is not limited to MR imaging systems but also covers MR systems in general.

1. A magnetic resonance imaging system comprising:
a magnet system for acquiring magnetic resonance spectroscopic data from an object of interest comprised within a polarizing magnetic field,
wherein the magnetic resonance imaging system comprises multiple pole faces for generating the polarizing magnetic field.

2. A system as claimed in claim 1, further comprising an indicator for indicating a region for acquiring the magnetic resonance spectroscopic data, said region being comprised within the polarizing magnetic field.

3. A system as claimed in claim 1, further comprising a positioner for positioning at least a part of the object of interest within the region for acquiring the magnetic resonance spectroscopic data.

4. A method of acquiring magnetic resonance spectroscopic data, the method comprising:
generating a polarizing magnetic field, and
acquiring magnetic resonance spectroscopic data from an object of interest comprised within the polarizing magnetic field,
wherein the polarizing magnetic field is generated between multiple pole faces of a magnetic resonance imaging system.

5. A method as claimed in claim 4, the method further comprising indicating a region for acquiring the magnetic resonance spectroscopic data, said region being comprised within the polarizing magnetic field.

6. A method as claimed in claim 4, the method further comprising positioning at least a part of the object of interest within the region for acquiring the magnetic resonance spectroscopic data.

7. A computer program product to be loaded by a computer arrangement comprising a processing unit and a memory, the computer program product comprising instructions for acquiring magnetic resonance spectroscopic data from a magnetic resonance imaging system and, after being loaded, enabling the magnetic resonance imaging system to carry out the following tasks:
generating a polarizing magnetic field, and
acquiring magnetic resonance spectroscopic data from an object of interest comprised within the polarizing magnetic field,
wherein the polarizing magnetic field is generated between multiple pole faces of the magnetic resonance imaging system.
